(19) United States
(12) Patent Application Publication
(10) Pub. No.: US 2019/0231994 A1
(43) Pub. Date: Aug. 1, 2019

(54) SPACER CHAMBER HAVING TWO AEROSOL PORTS

(71) Applicant: Diego Jaroslavsky, Ciudad Autonoma de Buenos Aires (AR)

(72) Inventor: Diego Jaroslavsky, Ciudad Autonoma de Buenos Aires (AR)

(21) Appl. No.: 16/383,741

(22) Filed: Apr. 15, 2019

Related U.S. Application Data
(63) Continuation-in-part of application No. 15/637,057, filed on Jun. 29, 2017.
Application No. 15/637,057 is a continuation-in-part of application No. 14/458,297, filed on Aug. 13, 2014, now abandoned.

(51) Int. Cl. A61M 15/00

(52) U.S. Cl. CPC A61M 15/009; A61M 15/0086

(57) ABSTRACT

A spacer chamber having a body with a base end; a tip end; a popoid section located between the base end and the tip end; a first aerosol port located on a first side of the body and near the tip end; a second aerosol port located on a second side of the body and near the tip end, the first side is opposite to the second side. Several therapeutic spray agents are simultaneously applied to a patient by the spacer chamber via the each one of the first aerosol port and the second aerosol port.

### CROSS-REFERENCE TO RELATED APPLICATIONS

This application is a continuation-in-part of patent application Ser. No. 15/637,057, FILED Jun. 29, 2017, entitled SPACER CHAMBER, pending, which is a continuation-in-part of patent application Ser. No. 14/458,297, filed Aug. 13, 2014, entitled SPACER CHAMBER, abandoned, the disclosure of both are incorporated herein by reference.

### FIELD OF THE INVENTION

The present invention refers to a spacer chamber that helps to disperse a medical drug to a patient. More specifically, the present invention relates to a spacer chamber having two aerosol ports.

### BACKGROUND INFORMATION

A series of spacer chambers having different shapes and mechanisms are commercially known to facilitate the pharmaceutical drug administration by respiratory inhalation. The majority of them being variations of the ones used in children or adults.

Many types of spacer chambers have been developed throughout the years. The known spacer chambers have different volumes and sizes. Unfortunately, a lot of the known chambers are difficult to transport in the case of being personally used.

Collapsible spacer chambers have been developed, with different types of structures, for example, including bellows (accordion shape) or with rigid rings. Unfortunately, they are difficult to properly extend, especially at the time the patient needs the medication.

In addition, the spacer chambers of the prior art are made of plastic, which is a material that generates a lot of static electricity and attracts the aerosol particles. To prevent this problem, the prior art created spacer chamber made of stainless steel. The prior art also added additives that do not charge with static electricity to prevent the adhesion of particles to the stainless-steel chambers.

### SUMMARY OF THE INVENTION

A spacer chamber having a body with a base end; a tip end; a popoid section located between the base end and the tip end; a first aerosol port located on a first side of the body and near the tip end; a second aerosol port located on a second side of the body and near the tip end, the first side is opposite to the second side. Several therapeutic spray agents are simultaneously applied to a patient by the spacer chamber via the each one of the first aerosol port and the second aerosol port.

### BRIEF DESCRIPTION OF THE DRAWINGS

FIG. 1 is a perspective view of the spacer chamber according to the prior art;

FIG. 2 is a side view of the spacer chamber according to an embodiment of the present invention;

FIG. 3 is a front view of the spacer chamber of FIG. 2;

FIG. 4 is a side view of the spacer chamber of FIG. 2 in an extended position;

FIG. 5 is a side view of the spacer chamber of FIG. 2 in a semi-folded position;

FIG. 6 is a side view of the spacer chamber according to an embodiment of the present invention including the chamber joined by their bases and also showing the chamber in an extended position;

FIG. 7 is a side view of the spacer chamber of FIG. 6 showing the chamber in a collapsed position;

FIG. 8 shows a side view of the spacer chamber according to one embodiment of the present invention showing a spacer chamber for outpatient treatment showing the chamber in an extended position;

FIG. 9 shows a top view of the spacer chamber of FIG. 8 showing the chamber in a collapsed position;

FIG. 10 shows a cross-sectional view of the spacer chamber taken along line A-A′ of FIG. 9 showing the chamber in a collapsed position;

FIG. 11 shows a side view of the spacer chamber according to one embodiment of the present invention showing a spacer chamber in use with a mechanical ventilation;

FIG. 12 shows a left side view of the spacer chamber of FIG. 11;

FIG. 13 shows a top view of the spacer chamber of FIG. 11;

FIG. 14 shows a cross-sectional view of the spacer chamber taken along line C-C of FIG. 13;

FIG. 15 shows a top view of the spacer chamber according to an embodiment of the present invention;

FIG. 16 is a cross-sectional view of the spacer chamber of FIG. 15 taken along line A-A′ showing the extended chamber and the mask connected to the chamber;

FIG. 17 is a top view of the spacer chamber according to an embodiment of the present invention showing the extended chamber in use with a mechanical ventilation and including a threaded base end and a threaded tip end;

FIG. 18 shows a cross-sectional view of the spacer chamber of FIG. 17 taken along line A-A′;

FIG. 19 is a side view of the spacer chamber according to an embodiment of the present invention showing the extended spacer chamber including a flap to extend the chamber, a thread end, and orifices in the threaded end;

FIG. 20 shows a top view of the spacer chamber of FIG. 19;

FIG. 21 is a top view of the spacer chamber according to an embodiment of the present invention showing the aerosol chamber with two aerosol ports connected to a corresponding aerosol device;

FIG. 22 shows a cross-sectional view of the spacer chamber of FIG. 21 taken along line A-A′;

FIG. 23 is a top view of the spacer chamber according to an embodiment of the present invention showing the aerosol chamber having an aerosol port having an aerosol device and a nebulizer port; and

FIG. 24 shows a cross-sectional view of the spacer chamber of FIG. 23 taken along line A-A′.

### DETAILED DESCRIPTION OF THE INVENTION

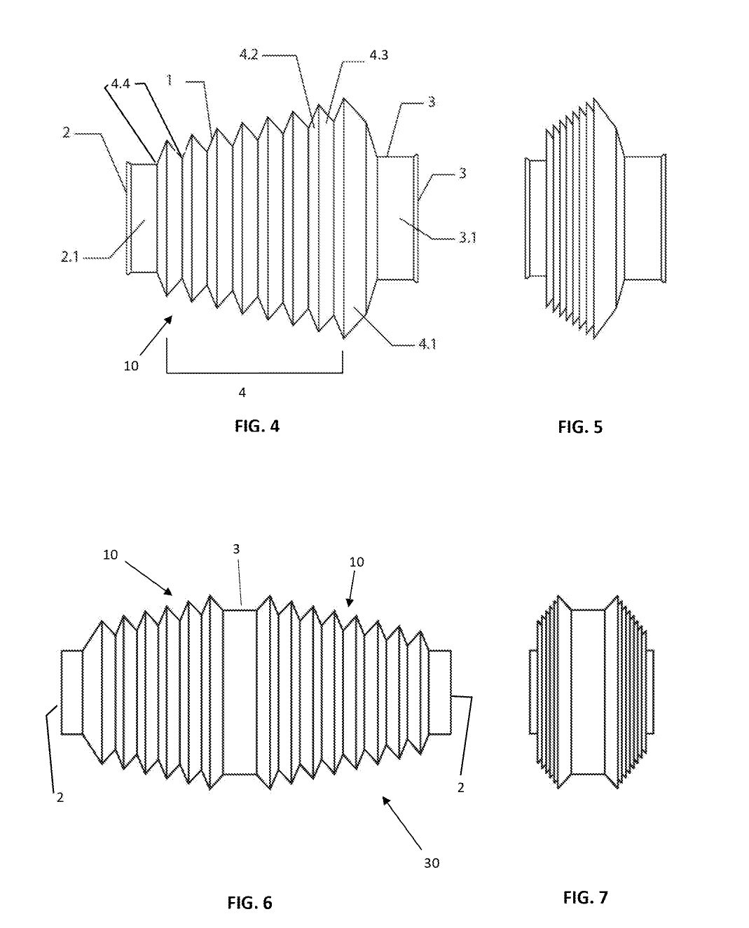

FIGS. 2 to 5 illustrate a spacer chamber 10 including a one-piece body 1 having a base end 3, a tip end 2, and a popoid section 4. The one-piece body 1 has a shape of a cone with the diameter 3.1 of the base end 3 larger than the diameter 2.1 of the tip end 2.

The body 1 is of one-piece construction and is preferably formed from a suitable anti-static material, such as polypropylene.

The popoid section 4 includes a plurality of popoids 4.1. Each popoid 4.1 includes a first set of frustoconical portions 4.2 each one having a big end 4.2.1 and a small end 4.2.2, and a second set of frustoconical shaped portions (4.3) each having a big end 4.3.1 and a small end 4.3.2. Each big end 4.2.1 of the first set of frustoconical shaped portions (4.2) faces opposite to the big end 4.3.1 of the corresponding second set of frustoconical portions 4.3 to form a corrugation 4.4 in the body 1. Each small end 4.2.2 of the first set of frustoconical shaped portions (4.2) contact the small end 4.3.2 of the adjacent popoid (4.1).

The first and second set of frustoconical portions 4.2, 4.3 of the corrugation 4.4 near to the base end 3 have a greater diameter than the diameter of the first and second set of frustoconical portions 4.2, 4.3 of the popoid 4.1 near to the tip end 2.

When the tip end 2 is moved toward the base end 2, the frustoconical portions 4.3 maintain their shape, while the frustoconical portions 4.2 become inverted to fit within the associated frustoconical portions 4.3 Thus, each popoid 4.1 will collapse upon itself. In addition, each popoid 4.1 beginning from the tip end 2 to the base end 3, will collapse completely inside the adjacent popoid 4.1 shortening the length of the body 1.

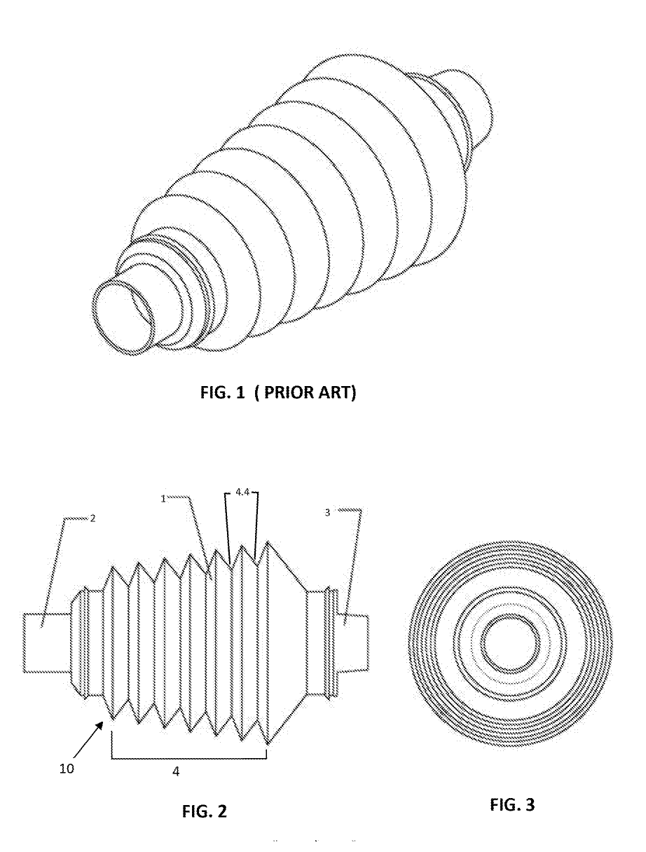

The collapsing of each one of the frustoconical portions 4.2 into the corresponding frustoconical portions 4.3, allows the popoid 4.1 to keep their mechanical strength longer since the material forming the popoid 4.1 is expanded to a lesser degree than the popoid of the prior art shown on FIG. 1.

FIGS. 6-7 shows a spacer chamber 30 according to an embodiment of the present invention including to chambers 10 joined by their bases.

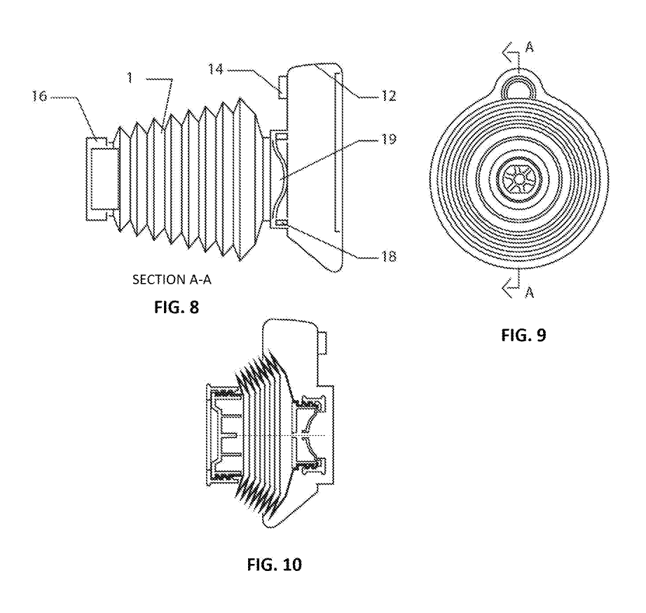

FIGS. 8-10 show the spacer chamber 10 according to the present invention showing a spacer chamber for use during outpatient treatment. A mask 12 for the patient is connected to the base end 3. The mask 12 includes a threaded end 18 to connect to the base end 3, an exhalation valve 14, and an aspiration valve 19. A threaded support 16 for aerosol is connected to the tip end 2.

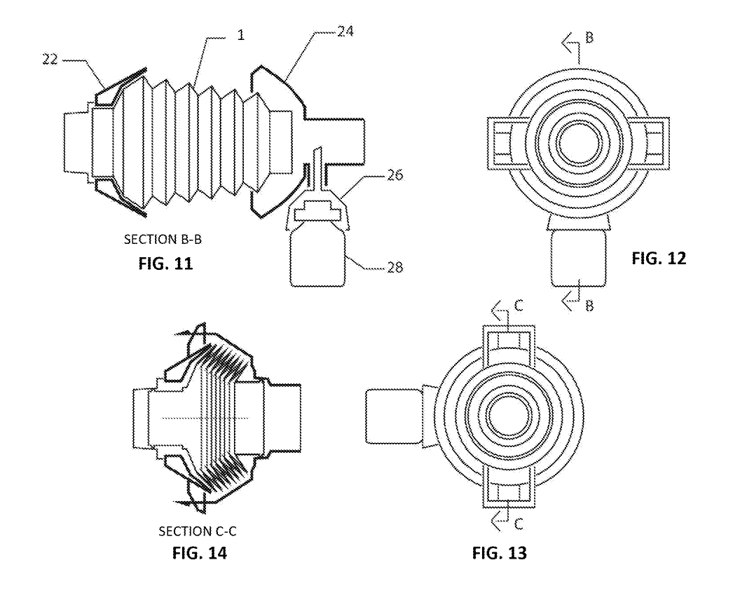

FIGS. 11-14 show the spacer chamber 10 of the present invention in use with a mechanical ventilation. A female connector 22 is connected to the tip end 2 of the body 1. The female connector 22 may include clamps to prevent the chamber to extend because of the pressure of the ventilator. A male connector 24 is connected to the base end 3 for air input into the chamber. The male connector 24 includes clamps for helping to hold the aerosol input device 26 that receives the aerosol device 28.

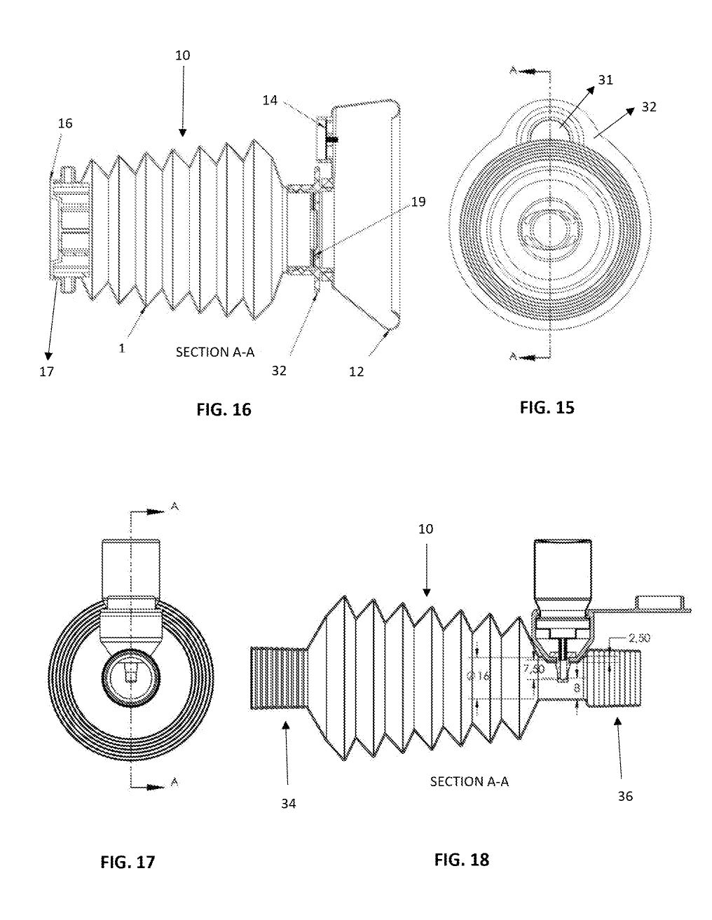

FIGS. 15-16 show the spacer chamber 10 including a flap 32 built into the base end 2. The flap 32 serves as a handle to help the patient to manipulate the extension of the body 1. The flap 32 is built into the base end 2 during the manufacturing process. Because the flap 32 is built into the base end, the flap 32 is made of the same material as the one-piece body 1. The flap 32 may include a hole 31 adapted to accommodate a finger of the patient during the extension of the body 1.

FIGS. 17 and 18 show a spacer chamber according to an embodiment of the present invention showing the extended chamber in use with a mechanical ventilation and including a threaded base end 34 and a threaded tip end 36.

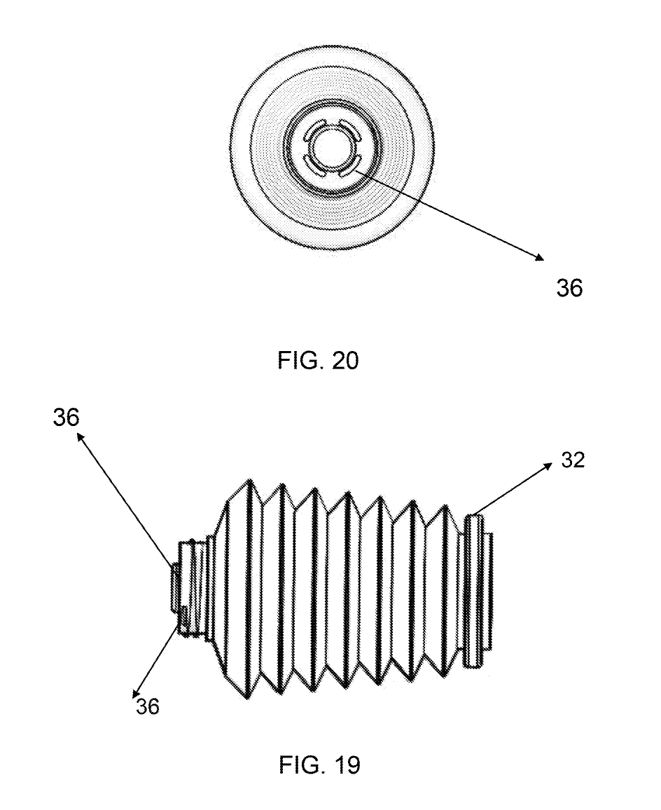

FIGS. 19-20 show the spacer chamber 10 including the flap 32, a threaded tip end, and orifices 36 in the threaded tip end 36. The orifices 36 allow the aerosol to pass through. In addition, the valve 14 covers the orifices 36 preventing blow inside the chamber.

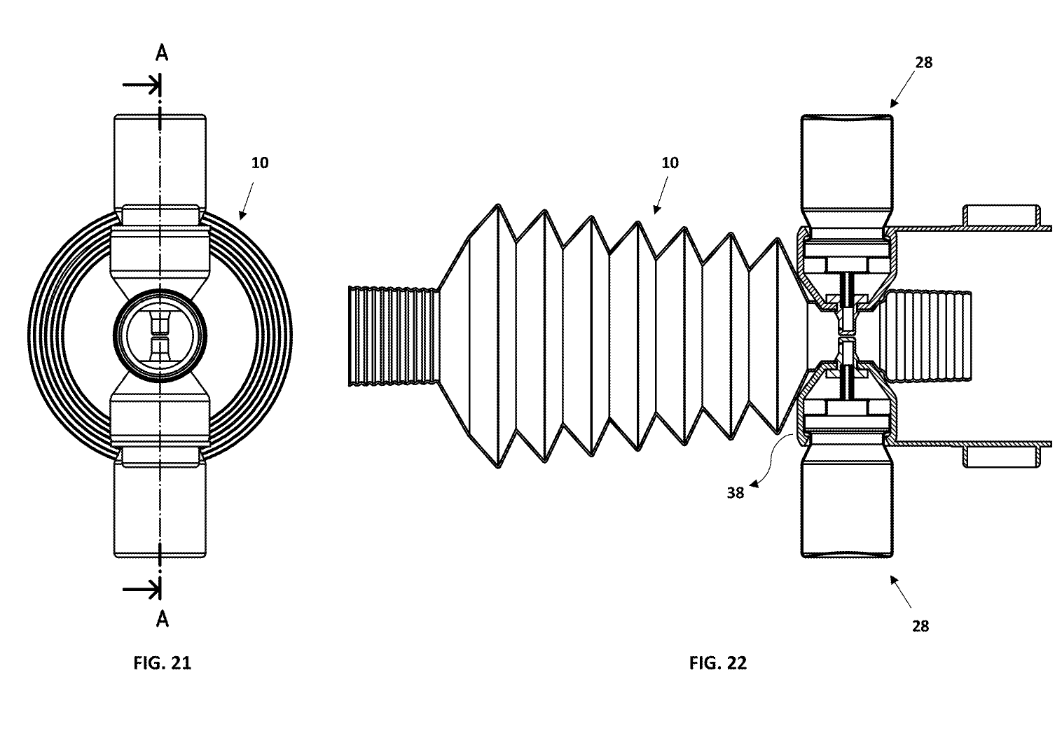

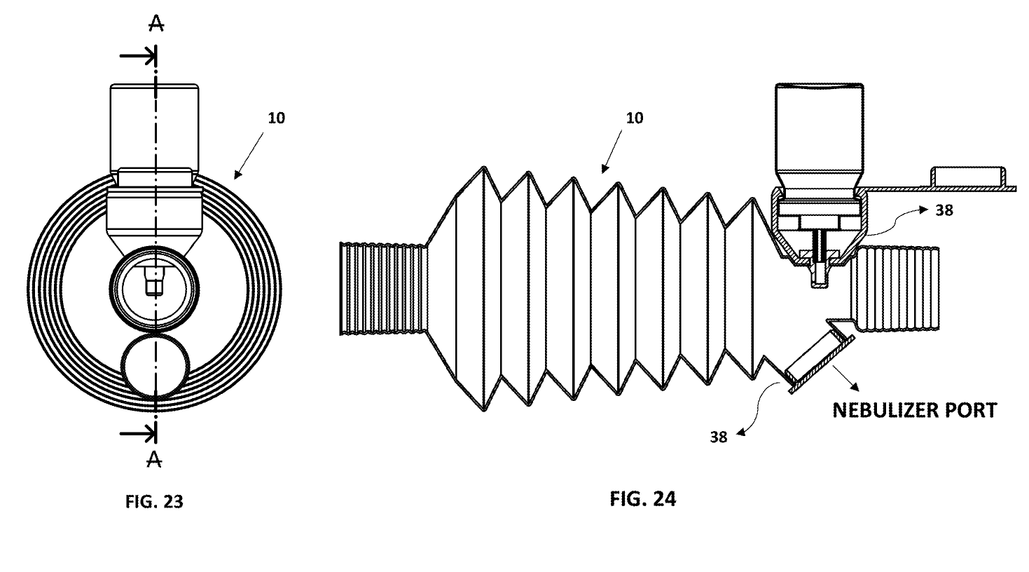

FIGS. 21-22 show the spacer chamber 10 according to an embodiment of the present invention showing the aerosol chamber with two aerosol ports 38. Each aerosol port is shown having connected to it a corresponding aerosol device 28. The connection is as previously shown.

Having two aerosol ports allows the patient to receive simultaneously two different types of medications.

FIGS. 23-24 show the spacer chamber according to an embodiment of the present invention showing the aerosol chamber having an aerosol port having an aerosol device connected to it and a nebulizer port.

1. A spacer chamber comprising:
a body having:
a base end;
a tip end;
a popoid section located between the base end and the tip end;
a first aerosol port located on a first side of the body and near the tip end;
a second aerosol port located on a second side of the body and near the tip end, the first side is opposite to the second side;
wherein several therapeutic spray agents are simultaneously applied to a patient by the spacer chamber via the each one of the first aerosol port and the second aerosol port.

2. The spacer chamber of claim 1, wherein the body is made of a flexible material.

3. The spacer chamber of claim 1, wherein the base end and the tip end are threaded.

4. The spacer chamber of claim 1, wherein the base end includes a built-in flap having a hole, the hole is adapted to receive a finger of a user to help in expanding the body.
